(19) United States
(12) Patent Application Publication
(10) Pub. No.: US 2016/0296101 A1
(43) Pub. Date: Oct. 13, 2016

(54) VARIABLE HARDNESS ACTUATOR

(71) Applicant: OLYMPUS CORPORATION, Tokyo (JP)

(72) Inventors: Tatsuhiko OKITA, Tokyo (JP); Kaoru MATSUKI, Kawasaki-shi (JP)

(73) Assignee: OLYMPUS CORPORATION, Tokyo (JP)

(21) Appl. No.: 15/190,665

(22) Filed: Jun. 23, 2016

Related U.S. Application Data
(63) Continuation of application No. PCT/JP2014/079234, filed on Nov. 4, 2014.

(30) Foreign Application Priority Data
Dec. 26, 2013 (JP) 2013-269994

(51) Int. Cl. A61B 1/00; A61B 1/005; F16L 55/00

(52) U.S. Cl. CPC A61B 1/00078; F16L 55/00; A61B 1/005

(57) ABSTRACT

A variable hardness actuator includes a first flexible member to which a first electrode is fixed, a second flexible member disposed to be opposed to the first flexible member, a second electrode being fixed to the second flexible member, an insulating member configured to insulate the electrodes, a driving section configured to apply voltages to the electrodes, and an instructing section configured to instruct the driving section to apply a voltage. When the driving section applies the voltage according to the instruction from the instructing section, electrostatic attraction is generated between the electrodes and a frictional force is generated between the flexible members. The flexible members integrally harden against a bending force from an outside.

### CROSS REFERENCE TO RELATED APPLICATION

This application is a continuation application of PCT/JP2014/079234 filed on Nov. 4, 2014 and claims benefit of Japanese Application No. 2013-269994 filed in Japan on Dec. 26, 2013, the entire contents of which are incorporated herein by this reference.

### BACKGROUND OF THE INVENTION

1. Field of the Invention

The present invention relates to a variable hardness actuator that can change rigidity against a bending force.

2. Description of the Related Art

For example, when a tube is inserted into a lumen having a complicated shape, if the tube is flexible, deformation occurs when the tube collides with a bent shape portion of the lumen and the tube remains in a position of the bent shape portion. It is difficult to insert the tube deeper into the lumen.

Therefore, there has been proposed a variable hardness actuator that can change rigidity of a member against a bending force. As an example, the actuator is used in a field of an endoscope in order to improve an insertion property.

As an example of such a variable hardness actuator, for example, Japanese Patent No. 5124629 describes a hardness adjusting device that draws a wire attached to a distal end of a contact coil spring to thereby change a compressed state of the contact coil spring and changes hardness. Further, the publication describes a technique for alleviating force by providing an elastic body in a wire drawing mechanism taking into account the fact that large force is required for operation in drawing the wire. More specifically, drawing torque necessary for the drawing is shifted to a negative side using a torsion spring or a spiral spring as the elastic body. However, since there is a portion where the drawing torque is negative, an action of a negative drawing force is located by using a combination of a worm gear and a worm wheel.

### SUMMARY OF THE INVENTION

A variable hardness actuator according to an aspect of the present invention includes: a first flexible member; a second flexible member disposed to be opposed to the first flexible member; a first electrode fixed to the first flexible member; a second electrode fixed to the second flexible member to be opposed to the first electrode; an insulating member disposed between the first electrode and the second electrode and configured to insulate the first electrode and the second electrode; a driving section configured to apply voltages to the first electrode and the second electrode; and an instructing section configured to instruct the driving section to apply a voltage. When the driving section applies the voltage according to the instruction from the instructing section, electrostatic attraction is generated between the first electrode and the second electrode and a frictional force is generated between the first flexible member and the second flexible member. The first flexible member and the second flexible member integrally harden against a bending force from an outside.

### BRIEF DESCRIPTION OF THE DRAWINGS

FIG. 1 is a diagram showing a configuration of a variable hardness actuator in a first embodiment of the present invention;

FIG. 2 is a sectional view showing a state of the variable hardness actuator at the time when the variable hardness actuator is hardened in the first embodiment;

FIG. 3 is a side view showing a state of the variable hardness actuator applied with a bending force when the variable hardness actuator is not hardened in the first embodiment;

FIG. 4 is a side view showing a state of the variable hardness actuator applied with a bending force when the variable hardness actuator is hardened in the first embodiment;

FIG. 5 is a sectional view showing a configuration of a modification of the variable hardness actuator in the first embodiment;

FIG. 6 is a time chart showing a state of a change in hardness of the variable hardness actuator at the time when an applied voltage is changed in the first embodiment;

FIG. 7 is a diagram showing a configuration of a variable hardness actuator in a second embodiment of the present invention;

FIG. 8 is a plan sectional view showing the configuration of the variable hardness actuator in the second embodiment;

FIG. 9 is a block diagram showing a configuration of a modification of the variable hardness actuator in the second embodiment; and

FIG. 10 is a perspective view showing a configuration of electrodes in a variable hardness actuator in a third embodiment of the present invention.

### DETAILED DESCRIPTION OF THE PREFERRED EMBODIMENTS

Embodiments of the present invention are explained below with reference to the drawings.

### First Embodiment

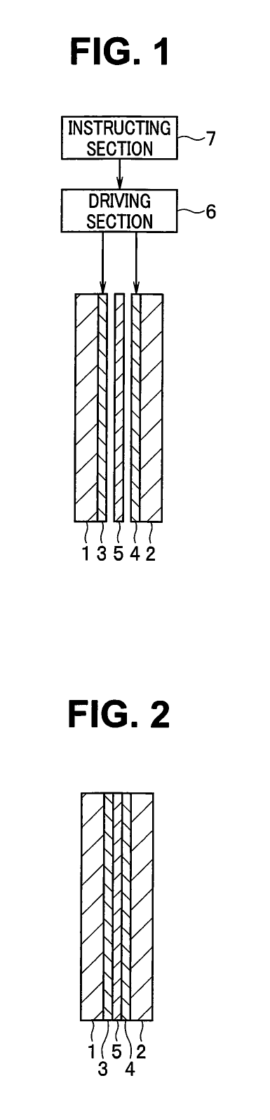

FIG. 1 to FIG. 6 are diagrams showing a first embodiment of the present invention. FIG. 1 is a diagram showing a configuration of a variable hardness actuator.

As shown in FIG. 1, the variable hardness actuator includes a first flexible member 1, a second flexible member 2, a first electrode 3, a second electrode 4, an insulating member 5, a driving section 6, and an instructing section 7.

The first flexible member 1 is a member configured to bend when a bending force is applied to the member. The first flexible member 1 is configured as, for example, a planar member.

The second flexible member 2 is a member disposed to be opposed to (directly opposed to or indirectly opposed to via another member or the like) the first flexible member 1 and configured to bend when a bending force is applied to the member. The second flexible member 2 is configured as, for example, a planar member.

In the present embodiment, the first flexible member 1 and the second flexible member 2 adopt a plane as an example of a shape assuming a planar shape and are formed as flat members. However, as indicated by other several examples in embodiments explained below, the first flexible member 1 and the second flexible member 2 may have a curved surface shape or may have a cylindrical shape and other shapes. Further, the first flexible member 1 and the second flexible member 2 are not limited to the planar shape and may have a linear shape or may be formed in the planar shape by, for example, combining linear members in, for example, a lattice shape.

The first electrode 3 is an electrode fixed to the first flexible member 1 and having a shape conforming to the planar shape of the first flexible member 1. In the present embodiment, a planar metal thin film (which may be foil or a thin plate) is adopted as the first electrode 3. Since the first electrode 3 is fixed to the first flexible member 1, when the first flexible member 1 receives a bending force and bends, the first electrode 3 also bends integrally with the first flexible member 1. Further, when electrostatic attraction acts on the first electrode 3 according to application of a voltage, the electrostatic attraction acts on the first flexible member 1 as well via the first electrode 3.

The second electrode 4 is an electrode fixed to the second flexible member 2 to be opposed to the first electrode 3 (however, in the configuration example shown in FIG. 1 and FIG. 2, opposed to the first electrode 3 explained above via the insulating member 5 rather than being directly opposed to the first electrode 3) and having a shape conforming to the planar shape of the second flexible member 2. In the present embodiment, a planar metal thin film (which may be foil or a thin plate) is adopted as the second electrode 4. Since the second electrode 4 is fixed to the second flexible member 2, when the second flexible member 2 receives a bending force and bends, the second electrode 4 also bends integrally with the second flexible member 2. Further, when electrostatic attraction acts on the second electrode 4 according to application of a voltage, the electrostatic attraction acts on the second flexible member 2 as well via the second electrode 4.

In the configuration example shown in FIG. 1 and FIG. 2, the first electrode 3 is fixed to a surface of the first flexible member 1 opposed to the second flexible member 2 and the second electrode 4 is fixed to a surface of the second flexible member 2 opposed to the first flexible member 1.

Note that the first electrode 3 and the second electrode 4 are not limited to electrodes provided over entire main surfaces of the first flexible member 1 and the second flexible member 2. Naturally, the first electrode 3 and the second electrode 4 may be locally provided only in a desired portion where hardness is expected to change.

The insulating member 5 is a member disposed between the first electrode 3 and the second electrode 4 and configured to insulate the first electrode 3 and the second electrode 4. In the present embodiment, an insulating sheet of a thin film (which may be a thin plate) is adopted. The insulating member 5 is formed as a thin film in order to reduce an inter-electrode distance between the first electrode 3 and the second electrode 4 as much as possible and increase electrostatic attraction acting during application of voltages as much as possible. Disposition and structure of the insulating member 5 are not limited as long as the insulating member 5 can keep the first electrode 3 and the second electrode 4 insulated. That is, the insulating member 5 may be present alone between the first electrode 3 and the second electrode 4 or may be provided as, for example, an insulating film on one or both of a surface of the first electrode 3 and a surface of the second electrode 4. Another member may perform the function of the insulating member 5 as explained below.

The driving section 6 applies voltages to the first electrode 3 and the second electrode 4 explained above. The driving section 6 includes, for example, a power supply and a transformer. Further, the driving section 6 in the present embodiment is capable of controlling a level of a voltage to be applied.

The instructing section 7 instructs the driving section 6 to apply a voltage. The instructing section 7 is configured by, for example, an operation switch. No particular power is required for operation of the instructing section 7. Light operation of the instructing section 7 is possible. Further, the instructing section 7 in the present embodiment is capable of instructing a level of the voltage that the instructing section 7 causes the driving section 6 to apply.

FIG. 2 is a sectional view showing a state of the variable hardness actuator at the time when the variable hardness actuator is hardened.

When the driving section 6 applies a voltage according to the instruction from the instructing section 7, electrostatic attraction is generated between the first electrode 3 and the second electrode 4. That is, force toward the second flexible member 2 is generated in the first flexible member 1. Conversely, force toward the first flexible member 1 is generated in the second flexible member 2. As shown in FIG. 2, the first electrode 3 and the second electrode 4 respectively come into contact with and adhere to one surface side and the other surface side of the insulating member 5 (electrostatic adhesion).

The attraction acting between the first flexible member 1 and the second flexible member 2 acts as a normal component of reaction. A frictional force is generated between the first flexible member 1 and the second flexible member 2 via the first electrode 3, the second electrode 4, and the insulating member 5.

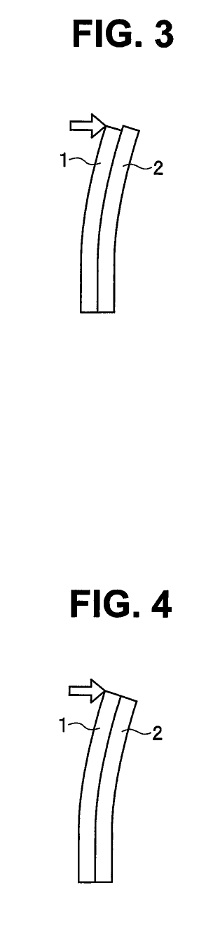

A change of the variable hardness actuator at the time when the variable hardness actuator receives a bending force from an outside in such a configuration is explained with reference to FIG. 3 and FIG. 4. Note that, in FIG. 3 and FIG. 4, it is assumed that the variable hardness actuator is fixed on a lower end side and a bending force from the left to the right indicated by a white arrow is applied to an upper end side.

First, FIG. 3 is a side view showing a state of the variable hardness actuator applied with a bending force when the variable hardness actuator is not hardened.

When voltages are not applied to the first electrode 3 and the second electrode 4 not to harden the first electrode 3 and the second electrode 4, a slip occurs between the first flexible member 1 and the second flexible member 2. Therefore, when a bending force is applied to the first flexible member 1 and the second flexible member 2 from the outside, the first flexible member 1 and the second flexible member 2 bend independently from each other and contact surfaces slide and shift. Consequently, a level difference occurs at a distal end as shown in FIG. 3. At this point, each of the first flexible member 1 and the second flexible member 2 is in a state in which a surface on a left side is stretched and a surface on a right side is compressed.

FIG. 4 is a side view showing a state of the variable hardness actuator applied with a bending force when the variable hardness actuator is hardened.

When voltages are applied to the first electrode 3 and the second electrode 4 to harden the first electrode 3 and the second electrode 4, the first flexible member 1 and the second flexible member 2 electrostatically adhere and bend integrally (that is, as one member having thickness obtained by adding up thickness of the first flexible member 1 and thickness of the second flexible member 2) when a bending force from the outside is applied to the first flexible member 1 and the second flexible member 2. The contact surfaces do not slide with a static frictional force (if the static frictional force is within a range of a maximum static frictional force or less). As shown in FIG. 4, a level difference does not occur at the distal end. At this point, surfaces on an opposed side (a surface on a right side of the first flexible member 1 and a surface on a right side of the second flexible member 2) are in an intermediate state between the stretch and the compression. However, the surface on the left side of the first flexible member 1 is in a greatly stretched state and the surface on the right side of the second flexible member 2 is in a greatly compressed state.

More specifically, if bending amounts are the same in the state shown in FIG. 3 and the state shown in FIG. 4, a stretch amount of the surface on the left side of the first flexible member 1 in the state shown in FIG. 4 is, for example, twice as large as a stretch amount of the surfaces on the left side of the first flexible member 1 and the second flexible member 2 in the state shown in FIG. 3. Similarly, a compression amount of the surface on the right side of the second flexible member 2 in the state shown in FIG. 4 is, for example, twice as large as a compression amount of the surfaces on the right side of the first flexible member 1 and the second flexible member 2 in the state shown in FIG. 3. Therefore, in the state shown in FIG. 4, stress larger than stress in the state shown in FIG. 3 occurs on insides of the first flexible member 1 and the second flexible member 2. That is, rigidity against a bending force increases.

In this way, the first flexible member 1 and the second flexible member 2 respectively behave as separate members against a bending force when voltages are not applied to the electrodes 3 and 4. However, when voltages are applied to the electrodes 3 and 4, the first flexible member 1 and the second flexible member 2 integrally behave as one member against the bending force. Therefore, the first flexible member 1 and the second flexible member 2 are hardened and hardness increases.

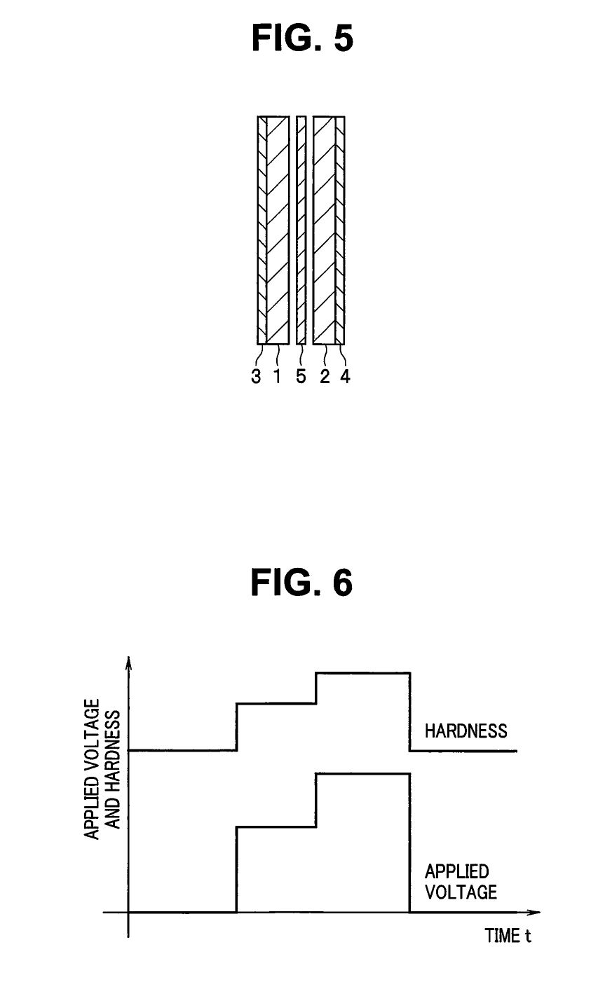

FIG. 5 is a sectional view showing a configuration of a modification of the variable hardness actuator.

In the configuration example shown in FIG. 1 and FIG. 2, in order to reduce the inter-electrode distance and increase the electrostatic attraction, the first electrode 3 is disposed on the surface of the first flexible member 1 opposed to the second flexible member 2 and the second electrode 4 is disposed on the surface of the second flexible member 2 opposed to the first flexible member 1 (that is, the electrodes 3 and 4 are respectively disposed on surfaces on a proximity side of the flexible members 1 and 2). However, the present invention is not limited to such a configuration.

That is, as shown in FIG. 5, even if the first electrode 3 is disposed on a surface of the first flexible member 1 on an opposite side of the second flexible member 2 and the second electrode 4 is disposed on a surface of the second flexible member 2 on an opposite side of the first flexible member 1 (that is, the electrodes 3 and 4 are respectively disposed on surfaces on a separated side of the flexible members 1 and 2), it is possible to generate a certain degree of electrostatic attraction.

Note that, in the case of the configuration shown in FIG. 5, if at least one of the first flexible member 1 and the second flexible member 2 is formed of an insulative material, the flexible member can perform a function of the insulating member 5 as well. As a result, it is possible to exclude the insulating member 5.

On the other hand, if the first flexible member 1 and the second flexible member 2 are formed of a conductive material under a condition that the insulating member 5 is present, the first flexible member 1 can serve as the first electrode 3 as well and the second flexible member 2 can serve as the second electrode 4 as well. Therefore, it is possible to exclude the first electrode 3 and the second electrode 4.

FIG. 6 is a time chart showing a state of a change in hardness of the variable hardness actuator at the time when an applied voltage is changed.

The variable hardness actuator in the present embodiment is capable of adjusting hardness by controlling levels of voltages applied to the first electrode 3 and the second electrode 4.

First, even when a voltage is not applied, a certain degree of hardness corresponding to materials forming the respective members, thickness, and the like is present in the variable hardness actuator.

Subsequently, when voltages are applied to the first electrode 3 and the second electrode 4, as the voltages are higher, electrostatic attraction acting between the first electrode 3 and the second electrode 4 also increases. Since the electrostatic attraction acts as a normal component of reaction, when the applied voltages increase, a maximum static frictional force, which could be generated between the first flexible member 1 and the second flexible member 2, also increases. That is, the first flexible member 1 and the second flexible member 2 are integrated and an upper limit value of force resisting a bending force from the outside increases. The hardness (solidity) in FIG. 6 indicates an upper limit value of such force resisting the bending force from the outside.

Note that, in the above explanation, the layers including sets of the electrodes and the flexible members are stacked in two layers. However, present invention is not limited to this. A configuration in which a larger number of layers are laid one on top of another may be adopted.

When a variable hardness actuator having certain thickness is configured by n layers, a bending force necessary for obtaining a certain bending amount is F∞Fn×n̂2 (a sign “̂2” represents a square) when the bending force at the time when electrostatic adhesion does not occur is represented as Fn and the bending force at the time when the electrostatic adhesion occurs is represented as F. That is, the bending force proportional to the square of the number of stacks is necessary.

Therefore, if the variable hardness actuator is configured by a large number of layers, there is an advantage that it is possible to increase a dynamic range of a hardness change.

According to the first embodiment explained above, it is possible to increase rigidity against a bending force by integrating the first flexible member 1 and the second flexible member 2 with the electrostatic attraction generated by applying a voltage between the first electrode 3 and the second electrode 4.

It is possible to adjust a degree of hardening to a desired degree by controlling levels of the voltages applied to the first electrode 3 and the second electrode 4.

When the first electrode 3 is fixed to the surface of the first flexible member 1 on the side opposed to the second flexible member 2 and the second electrode 4 is fixed to the surface of the second flexible member 2 on the side opposed to the first flexible member 1, a distance between the first electrode 3 and the second electrode 4 can be reduced as much as possible. Therefore, when a voltage to be applied is the same, stronger electrostatic attraction can be generated compared with when the distance between the electrodes is larger. It is possible to more effectively obtain high hardness.

Further, when at least one of the first flexible member 1 and the second flexible member 2 serves as the insulating member 5 as well, there is an advantage that it is unnecessary to separately provide the insulating member 5.

In addition, when the first flexible member 1 and the second flexible member 2 are formed of the conductive material, the first flexible member 1 and the second flexible member 2 can serve as the electrodes 3 and 4 as well. Therefore, it is unnecessary to provide the electrodes 3 and 4 separately from the flexible members 1 and 2. It is possible to reduce the number of components.

In this way, with the variable hardness actuator in the present embodiment, it is possible to lightly perform operation for changing hardness without depending on power.

### Second Embodiment

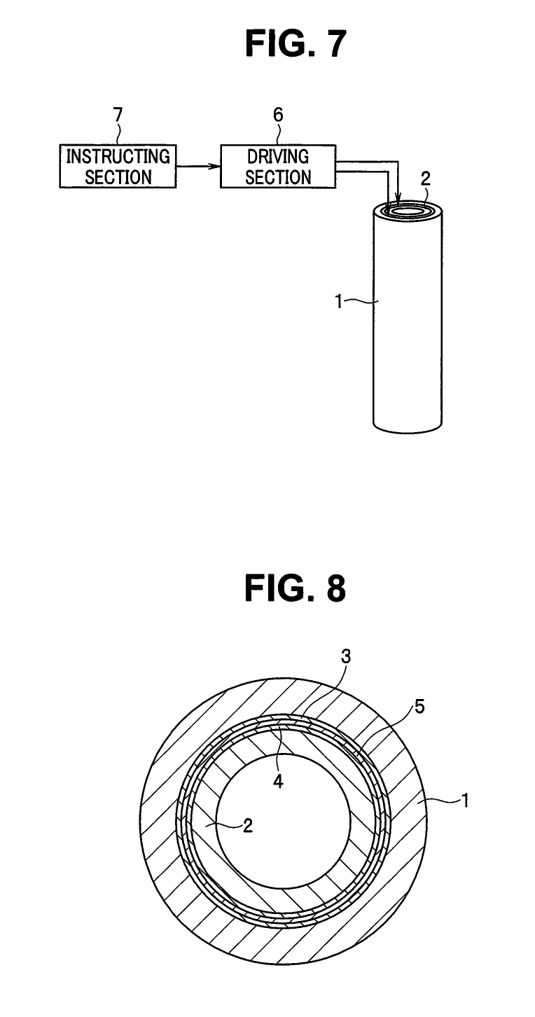

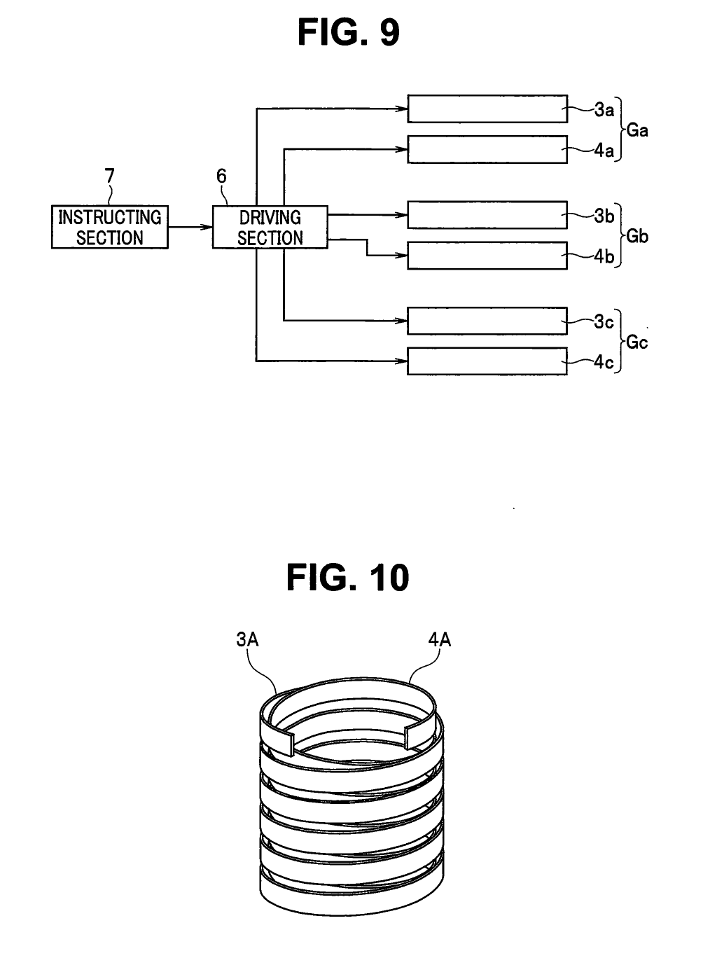

FIG. 7 to FIG. 9 show a second embodiment of the present invention. FIG. 7 is a diagram showing a configuration of a variable hardness actuator. FIG. 8 is a plan sectional view showing the configuration of the variable hardness actuator. FIG. 9 is a block diagram showing a configuration of a modification of the variable hardness actuator.

In the second embodiment, explanation is omitted as appropriate concerning portions same as the portions in the first embodiment by, for example, denoting the portions with the same reference numerals. Only differences are mainly explained.

In the first embodiment explained above, the variable hardness actuator is configured in the flat shape. However, in the case of the flat shape, hardness changes with respect to a bending force from a specific direction (e.g., a direction perpendicular to a flat principal plane). The hardness hardly changes with respect to a bending force from a direction different from the specific direction (e.g., a direction parallel to the flat principal plane) even if voltages are applied to the electrodes.

Therefore, in the present embodiment, a variable hardness actuator is configured in a cylindrical shape to be capable of changing hardness with respect to bending forces from a more various directions.

That is, as shown in FIG. 7, the first flexible member 1 is a member assuming a cylindrical shape.

The second flexible member 2 is a member assuming a cylindrical shape smaller in diameter than the first flexible member 1. An outer circumferential face of the second flexible member 2 is coaxially inserted through and disposed on an inner circumference side of the first flexible member 1 to be opposed to an inner circumferential face of the first flexible member 1.

Further, as shown in FIG. 8, the first electrode 3 has a shape conforming to the cylindrical shape of the first flexible member 1. More specifically, the first electrode 3 in the present embodiment is a cylindrical metal thin film fixed to the inner circumferential face of the first flexible member 1.

The second electrode 4 has a shape conforming to the cylindrical shape of the second flexible member 2. More specifically, the second electrode 4 in the present embodiment is a cylindrical metal thin film fixed to the outer circumferential face of the second flexible member 2.

Note that, in FIG. 7 and FIG. 8, an example is shown in which the electrodes 3 and 4 are disposed on the surfaces on the proximity side of the flexible members 1 and 2. However, as in the example shown in FIG. 5 in the first embodiment explained above, it is also possible to fix the first electrode 3 to the cylindrical outer circumferential face of the first flexible member 1 and fix the second electrode 4 to the cylindrical inner circumferential face of the second flexible member 2 (that is, dispose the electrodes 3 and 4 on the surfaces on the separated side of the flexible members 1 and 2).

In order to keep the first electrode 3 and the second electrode 4 insulated, the insulating member 5 is also formed as, for example, a thin film assuming a cylindrical shape and disposed between the first electrode 3 and the second electrode 4. More specific examples of the insulating member 5 include an insulating coat formed on the surfaces of the first electrode 3 and the second electrode 4.

In the configuration in the present embodiment, as in the first embodiment explained above, electrostatic adhesion occurs according to application of voltages to the first electrode 3 and the second electrode 4. The first flexible member 1 and the second flexible member 2 adhere and the hardness of the first flexible member 1 and the second flexible member 2 increases. At this point, as in the first embodiment explained above, it is possible to change the hardness by controlling levels of voltages to be applied.

FIG. 9 is a diagram showing an example in which a plurality of electrodes are provided and respectively disposed in different portions of the variable hardness actuator.

More specifically, in FIG. 9, an example is shown in which three electrode pairs are provided (however, it goes without saying that the number of electrode pairs is not limited to three). A first electrode pair Ga is configured by a first electrode 3a and a second electrode 4a. A second electrode pair Gb is configured by a first electrode 3b and a second electrode 4b. A third electrode pair Gc is configured by a first electrode 3c and a second electrode 4c.

The three electrode pairs Ga to Gc are respectively disposed in different positions along a longitudinal direction (e.g., an axial direction) of the first flexible member 1 and the second flexible member 2 assuming the cylindrical shape.

The driving section 6 is individually connected to each of the three electrode pairs Ga to Gc. Voltages are independently applicable to the respective electrode pairs Ga to Gc (that is, presence or absence of voltage application is independently controllable and levels of voltages to be applied is independently controllable).

Further, the instructing section 7 is capable of instructing the driving section 6 about which level of a voltage to apply to which of the three electrode pairs Ga to Gc.

By adopting the configuration shown in FIG. 9, there is an advantage that it is possible to change hardness of a desired portion along a longitudinal direction (e.g., an axial direction) of the variable hardness actuator. Furthermore, by differentiating levels of voltages applied to the three electrode pairs Ga to Gc from one another, it is possible to set hardness of each portion of the variable hardness actuator to desired hardness. Therefore, it is possible to realize, for example, a state in which a first portion is soft, a state in which a second portion has first hardness, and a state in which a third portion has second hardness different from the first hardness.

Note that such a configuration for varying the hardness of the desired portion can be applied not only to the cylindrical variable hardness actuator explained in the second embodiment but also to a variable hardness actuator having any shape.

According to such a second embodiment, it is possible to achieve effects substantially the same as the effects of the first embodiment. Further, since the first flexible member 1 and the second flexible member 2 are formed in the concentric cylindrical shape, irrespective of from which direction of the cylindrical circumferential surface a bending force is applied, rigidity against the bending force can be set substantially the same. Since the variable hardness actuator is formed in the cylindrical shape, it is also possible to dispose other members in a space on the inner circumference side of the second flexible member 2. Therefore, for example, the variable hardness actuator is a variable hardness actuator having a configuration suitable for an endoscope.

### Third Embodiment

FIG. 10 is a diagram showing a third embodiment of the present invention and is a perspective view showing a configuration of electrodes in a variable hardness actuator.

In the third embodiment, explanation is omitted as appropriate concerning portions same as the portions in the first and second embodiments by, for example, denoting the portions with the same reference numerals. Only differences are mainly explained.

When the first flexible member 1 and the second flexible member 2 are disposed in, for example, an endoscope, bending frequently occurs when the endoscope is inserted into a subject. Therefore, the present embodiment is contrived to make it possible to improve durability of electrodes even if such frequent bending occurs.

That is, a first electrode 3A is formed by spirally winding an elongated conductive thin plate along a circumferential surface, as a specific example, an inner circumferential face of the first flexible member 1 while shifting an axial direction position.

A second electrode 4A is formed by spirally winding an elongated conductive thin plate along a circumferential surface, as a specific example, an outer circumferential face of the second flexible member 2 while shifting an axial direction position.

In this case, as shown in FIG. 10, a winding direction of the first electrode 3A and a winding direction of the second electrode 4A are desirably opposite directions.

Note that, naturally, such a spiral electrode configuration is also applicable to, for example, respective electrodes configuring a plurality of electrode pairs shown in FIG. 9.

According to such a third embodiment, it is possible to achieve effects substantially the same as the effects of the first and second embodiments. Further, by forming the first electrode 3A and the second electrode 4A in the spiral shape, it is possible not to prevent flexibility of the first flexible member 1 and the second flexible member 2 as much as possible when voltages are not applied to the first flexible member 1 and the second flexible member 2.

The first electrode 3A and the second electrode 4A formed in the spiral shape are less easily broken even when the first electrode 3A and the second electrode 4A are bent. Further, since the first electrode 3A and the second electrode 4A themselves have a spring property, the first electrode 3A and the second electrode 4A not only are resistant to breakage or peeling off but also can relatively easily return to an original shape.

Further, since the winding direction of the first electrode 3A and the winding directions of the second electrode 4A are the opposite directions, there is an advantage that bending creases less easily occur even if a direction of application of a bending force is biased.

The present invention is not limited to the embodiments per se. In an implementation stage, the constituent elements can be modified and embodied in a range not departing from the spirit of the present invention. Modes of various inventions can be formed by appropriate combinations of the plurality of constituent elements disclosed in the respective embodiments. For example, several constituent elements may be deleted from all the constituent elements described in the embodiments. Further, the constituent elements described in different embodiments may be combined as appropriate. In this way, naturally, various modifications and applications are possible within the range not departing from the spirit of the invention.

What is claimed is:

1. A variable hardness actuator comprising:
a first flexible member;
a second flexible member disposed to be opposed to the first flexible member;
a first electrode fixed to the first flexible member;
a second electrode fixed to the second flexible member to be opposed to the first electrode;
an insulating member disposed between the first electrode and the second electrode and configured to insulate the first electrode and the second electrode;
a driving section configured to apply voltages to the first electrode and the second electrode; and
an instructing section configured to instruct the driving section to apply a voltage, wherein
when the driving section applies the voltage according to the instruction from the instructing section, electrostatic attraction is generated between the first electrode and the second electrode, a frictional force is generated between the first flexible member and the second flexible member, and the first flexible member and the second flexible member integrally harden against a bending force from an outside.

2. The variable hardness actuator according to claim 1, wherein
the first flexible member is a member of a cylindrical shape,
the second flexible member is a member of a cylindrical shape smaller in diameter than the first flexible member, the second flexible member being coaxially disposed on an inner circumferential face of the first flexible member such that an outer circumferential face of the second flexible member is opposed to an inner circumferential face of the first flexible member,
the first electrode has a shape conforming to the cylindrical shape of the first flexible member, and
the second electrode has a shape conforming to the cylindrical shape of the second flexible member.

3. The variable hardness actuator according to claim 1, wherein
an electrode pair formed by the first electrode and the second electrode is provided in plurality along a longitudinal direction of the first flexible member and the second flexible member,
the driving section is capable of independently applying a voltage to each of the plurality of electrode pairs, and
the instructing section is capable of instructing the driving section to apply the voltage to which of the plurality of electrode pairs.

4. The variable hardness actuator according to claim 1, wherein
the driving section is capable of controlling a level of a voltage to be applied,
the instructing section is capable of instructing a level of the voltage that the instructing section causes the driving section to apply, and
hardness can be adjusted by controlling levels of the voltages applied to the first electrode and the second electrode.

5. The variable hardness actuator according to claim 2, wherein
the first electrode is fixed to the inner circumferential face of the first flexible member,
the second electrode is fixed to the outer circumferential face of the second flexible member,
the insulating member is formed as a thin film, and
when the driving section applies the voltage, the first electrode and the second electrode respectively come into contact with one surface side and another surface side, and the frictional force between the first flexible member and the second flexible member is generated via the insulating member.

6. The variable hardness actuator according to claim 2, wherein
the first electrode is fixed to an outer circumferential face of the first flexible member,
the second electrode is fixed to an inner circumferential face of the second flexible member, and
at least one of the first flexible member and the second flexible member serves also as the insulating member.

7. The variable hardness actuator according to claim 2, wherein
the first electrode is formed by spirally winding an elongated conductive thin plate along a circumferential surface of the first flexible member, and
the second electrode is formed by spirally winding an elongated conductive thin plate along a circumferential surface of the second flexible member.

8. The variable hardness actuator according to claim 7, wherein a winding direction of the first electrode and a winding direction of the second electrode are opposite directions.

9. The variable hardness actuator according to claim 2, wherein
the first flexible member is formed of a conductive material and serves also as the first electrode, and
the second flexible member is formed of a conductive material and serves also as the second electrode.
